(19) United States
(12) Patent Application Publication
(10) Pub. No.: US 2026/0077026 A1
(43) Pub. Date: Mar. 19, 2026

(54) METHODS FOR TREATING CANCER WITH HYPERACTIVE ADAR ENZYMES

(71) Applicants: THE UNIVERSITY OF CHICAGO, Chicago, IL (US); MARINE BIOLOGICAL LABORATORY, Woods Hole, MA (US)

(72) Inventors: Joshua J.C. ROSENTHAL, Woods Hole, MA (US); María F. MONTIEL, Woods Hole, MA (US); Jeffrey A. HUBBELL, Chicago, IL (US); John-Michael E. WILLIFORD, Chicago, IL (US); Lisa R. VOLPATTI, Chicago, IL (US)

(73) Assignees: THE UNIVERSITY OF CHICAGO, Chicago, IL (US); MARINE BIOLOGICAL LABORATORY, Woods Hole, MA (US)

(21) Appl. No.: 19/109,299

(22) PCT Filed: Sep. 7, 2023
(86) PCT No.: PCT/US2023/073620

§ 371 Date: Mar. 6, 2025

Related U.S. Application Data
(60) Provisional application No. 63/404,231, filed on Sep. 7, 2022.

(51) Int. Cl. A61K 38/50; A61K 31/7105; A61K 35/76; A61K 45/06; A61P 35/00; C12N 7/04; C12N 15/113

(52) U.S. Cl. CPC A61K 38/50; A61K 31/7105; A61K 35/76; A61K 45/06; A61P 35/00; C12N 7/04; C12N 15/113; C12Y 305/04004; C12N 2310/11; C12N 2310/531; C12N 2750/14123; C12N 2750/14133

(57) ABSTRACT

The inventors hypothesized that the induction of a multitude of neoantigens in tumor cells, through hundreds of thousands of RNA editing events, would render tumors more recognizable by the patient's own T cells and that this could serve as an off-the-shelf therapeutic and could enhance the efficacy of immunotherapies. Example 1 provides evidence of therapeutic efficacy by locally delivering a hyperactive adenosine-to-inosine (A-to-I) RNA editing enzyme (ADAR) to induce neoantigens and to increase the effectiveness of immunotherapies. Accordingly, aspects of the disclosure relate to a method for treating cancer in a subject comprising administering a composition comprising a polypeptide comprising an adenosine deaminase RNA-specific binding protein (ADAR) or a nucleic acid encoding a polypeptide comprising an adenosine deaminase RNA-specific binding protein (ADAR), wherein the composition is administered in combination with an immunotherapy; and wherein the ADAR comprises a hyperactive ADAR.

### CROSS-REFERENCE TO RELATED APPLICATIONS

This application claims the benefit of priority to U.S. Provisional Patent Application Ser. No. 63/404,231, filed Sep. 7, 2022, hereby incorporated by reference in its entirety.

This invention was made with government support under NS064259, and NS087726 awarded by the National Institutes of Health. The government has certain rights in the invention.

### SEQUENCE LISTING

The application contains a Sequence Listing in compliance with ST.26 format and is hereby incorporated by reference in its entirety. Said Sequence Listing, created on Aug. 31, 2023 is named ARCDP0773WO.xml and is 15.9 kilobytes in size.

### BACKGROUND OF THE INVENTION

### I. Field of the Invention

This invention relates to the field of methods of cancer biology.

### II. Background

Immune checkpoint inhibitors (ICIs), such as antibodies against PD-1 or PD-L1, are said to “release the brakes” on the immune system to fight cancer [1]. To avoid immune detection, some tumors upregulate the expression of the checkpoint molecule PD-1. When the binding of PD-1 to its ligand on T cells is disrupted by ICIs, the immune system can recognize and attack the cancer cells [2]. However, ICIs only work well as a monotherapy in a small subset of cancer patients. Current approaches to further enhance the immunogenicity of tumors include oncolytic viruses, gene therapy, and neoantigen vaccines.

In 2015, the first oncolytic virus was approved by the U.S. Food and Drug Administration (FDA) for treatment of certain melanomas [3]. This therapy comprises a modified herpes simplex virus injected directly into cancerous lesions. The virus has been engineered to replicate specifically within tumor cells and to induce secretion of granulocyte-macrophage colony-stimulating factor to further promote immunogenicity. Other oncolytic viruses have been engineered to induce secretion of pro-inflammatory cytokines such as IL-7 and IL-12 and have been shown to enhance the effect of ICIs in preclinical combination studies [4, 5]. A major limitation of these viruses is their safety, specifically viral toxicity, off-target adverse effects, and potential unexpected consequences of genetic manipulation [6].

To enhance safety profiles for applications in gene delivery, adeno-associated viruses (AAVs) have been developed as non-pathogenic viruses that are non-replicating in the absence of helper viruses [7]. AAVs have been widely used for cancer gene therapies. Examples include their use to induce pro-inflammatory cytokines for enhanced immunogenicity, or to deliver tumor suppressor genes, anti-angiogenesis factors to inhibit vasculature formation and tumor growth, and antibodies to block signaling pathways [8]. However, the introduction of a single gene may not be sufficient to mount a robust immune response against the tumor.

As an alternative strategy, neoantigen vaccines have been designed to enhance the immunogenicity of new proteins arising from tumor mutations [9, 10]. Some neoantigens, such as those found in cancer driver genes, are shared across patients and cancer types. However, the vast majority of neoantigens that are MHC-restricted and immunogenic are patient-specific [11]. Therefore, much effort has been dedicated to identifying and vaccinating against these patient-specific neoantigens. Methods for identifying neoantigens include the direct identification of peptides from peptide-MHC complexes and the prediction of neoantigens from machine learning algorithms combined with next-generation sequencing [12]. While both of these methods have shown promise in neoantigen target identification, they are time and resource intensive and need to be individualized for each patient.

Once patient-specific neoantigens have been identified, there are two main strategies to vaccinate against them: adoptive cell therapy with dendritic cell vaccines or neoantigen-specific T cells and personalized vaccines against predicted neoantigens [9]. Personalized neoantigen vaccines can be composed of adjuvanted synthetic long peptides [15], mRNA [16, 17], or DNA encoding up to 20 patient-specific neoantigens. There are many ongoing clinical trials of neoantigen vaccines either alone or in combination with ICIs [10]. However, technical challenges in neoantigen identification and logistical challenges involving personalization remain. Furthermore, cancer cells could continue to evade the immune system through tumor escape mechanisms, and cells that do not contain the identified neoantigens may continue to proliferate in the process of immunoediting [19]. Therefore, there is a need in the art for additional cancer therapeutic strategies.

### SUMMARY

The inventors hypothesized that the induction of a multitude of neoantigens in tumor cells, through hundreds of thousands of RNA editing events, would render tumors more recognizable by the patient's own T cells and that this could serve as an off-the-shelf therapeutic and could enhance the efficacy of immunotherapies. Examples 1 and 2 provide evidence of therapeutic efficacy by locally delivering a hyperactive adenosine-to-inosine (A-to-I) RNA editing enzyme (ADAR) to induce neoantigens and to increase the effectiveness of immunotherapies. Accordingly, described is a method for treating cancer in a subject comprising administering a composition comprising a polypeptide comprising an adenosine deaminase RNA-specific binding protein (ADAR) or a nucleic acid encoding a polypeptide comprising an adenosine deaminase RNA-specific binding protein (ADAR), wherein the composition is administered in combination with an immunotherapy; and wherein the ADAR comprises a hyperactive ADAR. Also described is a method for increasing the efficacy of an immunotherapy in a subject comprising administering a composition comprising a polypeptide comprising an adenosine deaminase RNA-specific binding protein (ADAR) or a nucleic acid encoding a polypeptide comprising an adenosine deaminase RNA-specific binding protein (ADAR), wherein the composition is administered in combination with an immunotherapy; and wherein the ADAR comprises a hyperactive ADAR. Provided is a population of viral particles comprising: (i) viral particles comprising a nucleic acid encoding for a polypeptide comprising a hyperactive ADAR; and (ii) viral particles comprising a nucleic acid encoding for an antisense RNA. Also described are viral particles comprising a nucleic acid encoding for a polypeptide comprising a hyperactive ADAR and a nucleic acid encoding for an antisense RNA. Further provided are methods of making and using the viral particles.

The methods of the disclosure also relate to a method for treating cancer in a subject comprising administering viral particles of the disclosure to the subject, wherein the subject is one that has or will receive an immunotherapy. Further methods include a method for increasing the efficacy of an immunotherapy, the method comprising administering viral particles of the disclosure to the subject, wherein the subject is one that has or will receive an immunotherapy. Also described is a method for making a viral particle, the method comprising transfecting a packing cell with a nucleic acid encoding for a polypeptide comprising a hyperactive ADAR and/or a nucleic acid encoding for an antisense RNA; and collecting virus produced from the packaging cell.

The polypeptide may comprise or exclude a lamda phage N protein (λN). The λN may comprise the amino acid sequence of SEQ ID NO:2 or an amino acid sequence with at least 70% sequence identity to SEQ ID NO:2: NARTRRRERRAEKQAQWKAAN (SEQ ID NO: 2). The λN may comprise an amino acid sequence that has or has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:2. The polypeptide may comprise or exclude a SNAP tag.

The method may comprise or exclude administration of an antisense RNA or a DNA encoding an antisense RNA. The hyperactive ADAR may have an A-to-G whole genome editing index of greater than 0.5 or an A-to-G Alu element editing index of greater than 6. The hyperactive ADAR may have an A-to-G whole genome editing index of or of at least 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2, or any derivable range therein. The hyperactive ADAR may have an A-to-G Alu element editing index of or of at least 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10, or any derivable range therein. The editing index is a global measure of editing activity, quantifying the fraction (percent) of the DNA nucleotides in the whole genome or Alu elements exhibiting an A-to-G change compared to the reference genome. Methods for calculating the base editing index are known in the art and available at, for example github.com/a2iEditing/BEIndexer (see also, for example, Buchumenski et al., Genome Res. December 2021 vol. 31 no. 12 2354-2361, which is herein incorporated by reference.

The method may comprise or exclude administration of viral particle(s) encoding an antisense RNA. The viral particle encoding the antisense RNA may be the same as the viral particle encoding the hyperactive ADAR. The hyperactive ADAR and the antisense RNA may be encoded on the same viral vector. The antisense RNA may be one that does not have a hairpin. The antisense RNA may be one that does not have a BoxB hairpin and/or does not bind to the hyperactive ADAR. The antisense RNA may be one that lacks genomic targeting and is not complimentary to a genomic sequence. The antisense may be further defined as non-targeting. A non-targeting antisense is one that is not complimentary to a genomic sequence or a cancer antigen. The term genomic sequence refers to endogenous sequences. The antisense may comprise less than 10, 9, 8, 7, 6, 5, or 4 nucleotides (or any derivable range therein), such as contiguous nucleotides that are complimentary to a genomic sequence.

The antisense RNA may comprise at least one hairpin. The hairpin may be a BoxB hairpin. The antisense RNA may comprise, may comprise at least, or may comprise at most 1, 2, 3, or 4 hairpins. The one or more hairpins may comprise or exclude the nucleotide sequence of GGCCCTGAAAAAGGGCC (SEQ ID NO:9) (or the RNA equivalent thereof). The antisense RNA may comprise a nucleotide sequence that is complimentary to a cancer neoantigen. Exemplary neoantigens include Braf p. Val600Glu; KRAS p.Gly12Asp; KRAS p.Gly12Val; TP53 p.Arg175His; KRAS p.Gly12Asp; KRAS p.Gly12Val; HRAS//KRAS/NRAS pGln61Arg; KRAS p.Gly12Val; BRAF p.Val600Glu; and KRAS p.Gly12Asp. These and other neoantigens are described in the art (see, for example, Pearlman, A. H., Hwang. M. S., Konig, M. F. et al. Targeting public neoantigens for cancer immunotherapy. Nat Cancer 2, 487-497 (2021), which is herein incorporated by reference). The antisense RNA may be further defined as a targeting RNA. A targeting RNA comprises nucleotide sequences that are complimentary to genomic sequences, such as at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides or contiguous nucleotides that are complimentary to genomic sequences. The targeting RNA may comprise a stretch of nucleotides that are complimentary to a target, such as a genomic sequence or neoantigen, and hairpins inserted within the stretch of complimentary nucleotides. A targeting RNA may have, for example, at least or at most 5, 6, 7, 8, 9, 10, 11, 12, 13, 14, or 15 nucleotides complimentary to a target, such as a neoantigen, followed by a BoxB hairpin, followed by at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides or at most complimentary to a target, such as a neoantigen, followed by a second BoxB hairpin, followed by at least, at most, or exactly 5, 6, 7, 8, 9, 10, 11, 12, 13, 14, or 15 nucleotides complimentary to a target, such as a neoantigen.

The antisense RNA may comprise less than 90 nucleotides. The antisense RNA may comprise less than 60 nucleotides. The antisense may comprise, comprise at least, or comprise at most 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, or 200 nucleotides, or any derivable range therein.

The immunotherapy may comprise an immune checkpoint inhibitor (ICI), an immune agonist antibody, or nucleic acids encoding for an immune checkpoint inhibitor or an immune agonist antibody. The immunotherapy may comprise an immunotherapy described herein. The ICI or immune agonist antibody may comprise or exclude an anti-CD40 agonistic antibody, anti-PD1 blocking antibody, anti-PDL1 blocking antibody, anti-CTLA4 blocking antibody, or combinations thereof. The immunotherapy may comprise pro-inflammatory molecules or nucleic acids encoding for pro-inflammatory molecules. Pro-inflammatory molecule may comprise or exclude IL-2, IL-12, IL-15, and combinations thereof. The immunotherapy may comprise or exclude an adjuvant or a nucleic acid encoding an adjuvant. The immunotherapy may comprise viral particles comprising nucleic acids encoding for the immunotherapy.

The hyperactive ADAR polypeptide and/or antisense RNA or nucleic acid encoding such may be administered before the immunotherapy or at approximately the same time as the immunotherapy. The polypeptide and/or antisense RNA or nucleic acid(s) encoding the hyperactive ADAR and/or antisense RNA may be administered before the immunotherapy and within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days (or any derivable range therein) of the immunotherapy. The polypeptide and/or antisense RNA or nucleic acid(s) encoding the hyperactive ADAR and/or antisense RNA may be administered after the immunotherapy and within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days (or any derivable range therein) of the immunotherapy. The ICI therapy may comprise monotherapy or combination therapy. The ICI therapy may comprise an anti-PD1 antibody.

The ADAR may comprise or exclude an E448Q substitution. The ADAR may comprise ADAR2 with an E448Q substation or an ADAR with an E-Q substitution in the position corresponding to 448, wherein the corresponding position is determined by aligning the sequences with human ADAR2 and determining the amino acid that aligns with amino acid 448 of the human ADAR2 (SEQ ID NO:3). The ADAR may comprise ADAR2. The ADAR may comprise ADAR1, such as the human ADAR1. The ADAR1 may comprise a E1008Q substitution. The ADAR may comprise the amino acid sequence of SEQ ID NO: 1 or an amino acid sequence having at least 70% sequence identity to SEQ ID NO:1. The ADAR may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:1:

(SEQ ID NO: 1)
LHLDQTPSRQPIPSEGLQLHLPQVLADAVSRLVLGKFGDLTDNESSPHAR
RKVLAGVVMTTGTDVKDAKVISVSTGTKCINGEYMSDRGLALNDCHAEII
SRRSLLRFLYTQLELYLNNKDDQKRSIFQKSERGGFRLKENVQFHLYIST
SPCGDARIFSPHEPILEEPADRHPNRKARGQLRTKIESGEGTIPVRSNAS
IQTWDGVLQGERLLTMSCSDKIARWNVVGIQGSLLSIFVEPIYFSSIILG
SLYHGDHLSRAMYQRISNIEDLPPLYTLNKPLLSGISNAEARQPGKAPNF
SVNWTVGDSAIEVINATTGKDELGRASRLCKHALYCRWMRVHGKVPSHLL
RSKITKPNVYHESKLAAKEYQAAKARLFTAFIKAGLGAWVEKPTEQDQFS
LTPLV.

The ADAR may comprise the amino acid sequence of one of SEQ ID NO:1, 3-8, 10, or 11 or an amino acid sequence having at least 70% sequence identity to SEQ ID NO:1, 3-8, 10, or 11. The ADAR may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 1, 3-8, 10, or 11.

The ADAR may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:10: LHLDQTPSRQPIPSEGLQLHLPQVLADAVSRLVLGKFGDLTDNFSSPHARRKVLAGV VMTTGTDVKDAKVISVSTGTKCINGEYMSDRGLALNDCHAEIISRRSLLRFLYTQLEL YLNNKDDQKRSIFQKSERGGFRLKENVQFHLYISTSPCGDARIFSPHEPILEEPADRHP NRKARGQLRTKIESGQGTIPVRSNASIQTWDGVLQGERLLTMSCSDKIARWNVVGIQ GSLLSIFVEPIYFSSIILGSLYHGDHLSRAMYQRISNIEDLPPLYTLNKPLLSGISNAEAR QPGKAPNFSVNWTVGDSAIEVINATTGKDELGRASRLCKHALYCRWMRVHGKVPS HLLRSKITKPNVYHESKLAAKEYQAAKARLFTAFIKAGLGAWVEKPTEQDQFSLTPL V (SEQ ID NO:10). The ADAR may comprise or consist of the catalytic domain of the ADAR. The ADAR may comprise a human ADAR. The ADAR may comprise an ADAR selected from the following, or a functional fragment thereof:

(SEQ ID NO: 3)
MDIEDEENMSSSSTDVKENRNLDNVSPKDGSTPGPGEGSQLSNGGGGGPGRKRPLEE
GSNGHSKYRLKKRRKTPGPVLPKNALMQLNEIKPGLQYTLLSQTGPVHAPLFVMSV
EVNGQVFEGSGPTKKKAKLHAAEKALRSFVQFPNASEAHLAMGRTLSVNTDFTSDQ
ADFPDTLFNGFETPDKAEPPFYVGSNGDDSFSSSGDLSLSASPVPASLAQPPLPVLPPF
PPPSGKNPVMILNELRPGLKYDELSESGESHAKSFVMSVVVDGQFFEGSGRNKKLAK
ARAAQSALAAIFNLHLDQTPSRQPIPSEGLQLHLPQVLADAVSRLVLGKFGDLTDNFS
SPHARRKVLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSPCGDARIFSP
HEPILEEPADRHPNRKARGQLRTKIESGEGTIPVRSNASIQTWDGVLQGERLLTMSCS
DKIARWNVVGIQGSLLSIFVEPIYFSSIILGSLYHGDHLSRAMYQRISNIEDLPPLYTLN
KPLLSGISNAEARQPGKAPNFSVNWTVGDSAIEVINATTGKDELGRASRLCKHALYC
RWMRVHGKVPSHLLRSKITKPNVYHESKLAAKEYQAAKARLFTAFIKAGLGAWVE
KPTEQDQFSLTP.
(SEQ ID NO: 11)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQTGPVHAP
LFVMSVEVNGQVFEGSGPTKKKAKLHAAEKALRSFVQFPNASEAHLAMGRTLSVNT
DFTSDQADFPDTLFNGFETPDKAEPPFYVGSNGDDSFSSSGDLSLSASPVPASLAQPPL
PVLPPFPPPSGKNPVMILNELRPGLKYDELSESGESHAKSFVMSVVVDGQFFEGSGRN
KKLAKARAAQSALAAIFNLHLDQTPSRQPIPSEGLQLHLPQVLADAVSRLVLGKFGD
LTDNFSSPHARRKVLAGVVMTTGTDVKDAKVISVSTGTKCINGEYMSDRGLALNDC
HAEIISRRSLLRFLYTQLELYLNNKDDQKRSIFQKSERGGFRLKENVQFHLYISTSPCG
DARIFSPHEPILEEPADRHPNRKARGQLRTKIESGQGTIPVRSNASIQTWDGVLQGERL
LTMSCSDKIARWNVVGIQGSLLSIFVEPIYFSSIILGSLYHGDHLSRAMYQRISNIEDLP
PLYTLNKPLLSGISNAEARQPGKAPNFSVNWTVGDSAIEVINATTGKDELGRASRLCK
HALYCRWMRVHGKVPSHLLRSKITKPNVYHESKLAAKEYQAAKARLFTAFIKAGLG
AWVEKPTEQDQFSLTP
(SEQ ID NO: 4)
MASSTTGPLHMGTFFSIMGRRYKRRRKKRSERKDRNSLRQSRNPQKYFA
MDIEDEENMSSSSTDVKENRNLDNVSPKDGSTPGPGEGSQLSNGGGGGPGRKRPLEE
GSNGHSKYRLKKRRKTPGPVLPKNALMQLNEIKPGLQYTLLSQTGPVHAPLFVMSV
EVNGQVFEGSGPTKKKAKLHAAEKALRSFVQFPNASEAHLAMGRTLSVNTDFTSDQ
ADFPDTLFNGFETPDKAEPPFYVGSNGDDSFSSSGDLSLSASPVPASLAQPPLPVLPPF
PPPSGKNPVMILNELRPGLKYDELSESGESHAKSFVMSVVVDGQFFEGSGRNKKLAK
ARAAQSALAAIFNLHLDQTPSRQPIPSEGLQLHLPQVLADAVSRLVLGKFGDLTDNFS
SPHARRKVLAGVVMTTGTDVKDAKVISVSTGTKCINGEYMSDRGLALNDCHAEIISR
RSLLRFLYTQLELYLNNKDDQKRSIFQKSERGGFRLKENVQFHLYISTSPCGDARIFSP
HEPILEGSRSYTQAGVQWCNHGSLQPRPPGLLSDPSTSTFQGAGTTEPADRHPNRKA
RGQLRTKIESGEGTIPVRSNASIQTWDGVLQGERLLTMSCSDKIARWNVVGIQGSLLS
IFVEPIYFSSIILGSLYHGDHLSRAMYQRISNIEDLPPLYTLNKPLLSGISNAEARQPGK
APNFSVNWTVGDSAIEVINATTGKDELGRASRLCSLPLTTLQDYQAQRVP.
(SEQ ID NO: 5)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQTGPVHAP
LFVMSVEVNGQVFEGSGPTKKKAKLHAAEKALRSFVQFPNASEAHLAMGRTLSVNT
DFTSDQADFPDTLFNGFETPDKAEPPFYVGSNGDDSFSSSGDLSLSASPVPASLAQPPL
PVLPPFPPPSGKNPVMILNELRPGLKYDELSESGESHAKSFVMSVVVDGQFFEGSGRN
KKLAKARAAQSALAAIFNLHLDQTPSRQPIPSEGLQLHLPQVLADAVSRLVLGKFGD
LTDNFSSPHARRKVLAGVVMTTGTDVKDAKVISVSTGTKCINGEYMSDRGLALNDC
HAEIISRRSLLRFLYTQLELYLNNKDDQKRSIFQKSERGGFRLKENVQFHLYISTSPCG
DARIFSPHEPILEEPADRHPNRKARGQLRTKIESGEGTIPVRSNASIQTWDGVLQGERL
LTMSCSDKIARWNVVGIQGSLLSIFVEPIYFSSIILGSLYHGDHLSRAMYQRISNIEDLP
PLYTLNKPLLSGISNAEARQPGKAPNFSVNWTVGDSAIEVINATTGKDELGRASRLCK
HALYCRWMRVHGKVPSHLLRSKITKPNVYHESKLAAKEYQAAKVH
(SEQ ID NO: 6)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQTGPVHAP
LFVMSVEVNGQVFEGSGPTKKKAKLHAAEKALRSFVQFPNASEAHLAMGRTLSVNT
DFTSDQADFPDTLFNGFETPDKAEPPFYVGSNGDDSFSSSGDLSLSASPVPASLAQPPL
PVLPPFPPPSGKNPVMILNELRPGLKYDFLSESGESHAKSFVMSVVVDGQFFEGSGRN
KKLAKARAAQSALAAIFNLHLDQTPSRQPIPSEGLQLHLPQVLADAVSRLVLGKFGD
LTDNFSSPHARRKVLAGVVMTTGTDVKDAKVISVSTGTKCINGEYMSDRGLALNDC
HAEIISRRSLLRFLYTQLELYLNNKDDQKRSIFQKSERGGFRLKENVQFHLYISTSPCG
DARIFSPHEPILEGSRSYTQAGVQWCNHGSLQPRPPGLLSDPSTSTFQGAGTTEPADR
HPNRKARGQLRTKIESGEGTIPVRSNASIQTWDGVLQGERLLTMSCSDKIARWNVVG
IQGSLLSIFVEPIYESSIILGSLYHGDHLSRAMYQRISNIEDLPPLYTLNKPLLSGISNAE
ARQPGKAPNFSVNWTVGDSAIEVINATTGKDELGRASRLCKHALYCRWMRVHGKV
PSHLLRSKITKPNVYHESKLAAKEYQAAKARLFTAFIKAGLGAWVEKPTEQDQFSLT
P.
(SEQ ID NO: 7)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQTGPVHAP
LFVMSVEVNGQVFEGSGPTKKKAKLHAAEKALRSFVQFPNASEAHLAMGRTLSVNT
DFTSDQADFPDTLFNGFETPDKAEPPFYVGSNGDDSFSSSGDLSLSASPVPASLAQPPL
PVLPPFPPPSGKNPVMILNELRPGLKYDELSESGESHAKSFVMSVVVDGQFFEGSGRN
KKLAKARAAQSALAAIFNLHLDQTPSRQPIPSEGLQLHLPQVLADAVSRLVLGKFGD
LTDNFSSPHARRKVLAGVVMTTGTDVKDAKVISVSTGTKCINGEYMSDRGLALNDC
HAEIISRRSLLRFLYTQLELYLNNKDDQKRSIFQKSERGGFRLKENVQFHLYISTSPCG
DARIFSPHEPILEGSRSYTQAGVQWCNHGSLQPRPPGLLSDPSTSTFQGAGTTEPADR
HPNRKARGQLRTKIESGEGTIPVRSNASIQTWDGVLQGERLLTMSCSDKIARWNVVG
IQGSLLSIFVEPIYFSSIILGSLYHGDHLSRAMYQRISNIEDLPPLYTLNKPLLSGISNAE
ARQPGKAPNFSVNWTVGDSAIEVINATTGKDELGRASRLCKHALYCRWMRVHGKV
PSHLLRSKITKPNVYHESKLAAKEYQAAKVH.
(SEQ ID NO: 8)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQTGPVHAP
LFVMSVEVNGQVFEGSGPTKKKAKLHAAEKALRSFVQFPNASEAHLAMGRTLSVNT
DFTSDQADFPDTLFNGFETPDKAEPPFYVGSNGDDSFSSSGDLSLSASPVPASLAQPPL
PVLPPFPPPSGKNPVMILNELRPGLKYDELSESGESHAKSFVMSVVVDGQFFEGSGRN
KKLAKARAAQSALAAIFNLHLDQTPSRQPIPSEGLQLHLPQVLADAVSRLVLGKFGD
LTDNFSSPHARRKVLAGVVMTTGTDVKDAKVISVSTGTKCINGEYMSDRGLALNDC
HAEIISRRSLLRFLYTQLELYLNNKDDQKRSIFQKSERGGFRLKENVQFHLYISTSPCG
DARIFSPHEPILEEPADRHPNRKARGQLRTKIESGEGTIPVRSNASIQTWDGVLQGERL
LTMSCSDKIARWNVVGIQGSLLSIFVEPIYFSSIILGSLYHGDHLSRAMYQRISNIEDLP
PLYTLNKPLLSGISNAEARQPGKAPNFSVNWTVGDSAIEVINATTGKDELGRASRLCK
HALYCRWMRVHGKVPSHLLRSKITKPNVYHESKLAAKEYQAAKVH.

The cancer may comprise melanoma. The cancer may be a cancer described herein. The cancer may comprise an immunologically cold tumor. The term “immunologically cold tumor” refers to a tumor that is not likely to trigger a strong immune response. Cold tumors tend to be surrounded by cells that are able to suppress the immune response and keep T cells from attacking the tumor cells and killing them. The cancer may comprise a cancer that is resistant to immunotherapy. The cancer may comprise, breast, ovarian, prostate, pancreatic, or brain (glioblastoma) cancer. The composition may be administered by intratumoral or peritumoral injection. The composition may be administered by a route of administration described herein. The subject may be further defined as a human subject. The subject may be a mammalian subject. The subject may be a human, rat, pig, horse, mouse, rabbit, dog, cat, laboratory animal, or domesticated animal. The ADAR may be a human ADAR or a derivative thereof. The method may comprise administering a viral particle comprising a nucleic acid encoding a chimeric polypeptide comprising a λN and an ADAR. The viral particle may comprise an adeno-associated viral (AAV) particle or a derivative thereof. The viral particle may comprise a lentiviral particle, or a derivative thereof. The AAV serotype may be DJ. The viral particle may comprise an oncolytic viral particle.

The ratio of viral particles encoding the hyperactive ADAR polypeptide to viral particles encoding the antisense RNA may be from about 1:2 to 1:10. The ratio of viral particles encoding the hyperactive ADAR polypeptide to viral particles encoding the antisense RNA may be 1:7. The ratio of viral particles encoding the hyperactive ADAR polypeptide to viral particles encoding the antisense RNA may be 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 to 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10 (or any derivable range therein). The antisense RNA may comprise an antisense RNA and wherein the antisense RNA directs a genomic editing event of a target gene. The target gene may comprise an oncogene, a tumor suppressor, a metabolic gene, a cytokine, or a growth factor. Target genes include or exclude, for example, Ras family genes, such as KRAS, HRAS, NRAS, c-myc, p53, RB1, hTERT, VHL, BRCA1, BRCA2, GNA13, metabolic genes, such as HIFla, GLUT1 transporters, glutaminase, arginase, alanine, bransched chain amino acids, fatty acids, effectors, such as cytokines, IL-24, MDA, IL-6, and growth factors such as EGFR. Target genes may also include tumor suppressors such as RB1, TP53, CDKN2A, APC, MLH1, MSH2, MSH6, WT1, WT2, NF1, NF2, or VHL or oncogenes such as abl, akt, bcl-2, D1, E2A/pbx1, erbB2, gip, gli, gsp, hox11, cmyc, L-myc, N-myc, PDGFR, PML/RARa, rasH, rasK, rasN, ret, and SMO.

The viral particles may comprise tumor targeting viral particles. The term “tumor targeting viral particles” refers to viral particles engineered to preferentially or specifically target tumor cells. Examples of methods for making and designing such viral particles are known in the art (see, for example, Santiago-Ortiz et al., J Control Release. 2016 Oct. 28; 240:287-301, which is specifically incorporated by reference). The viral particles may comprise adeno-associated viral particles. The method may comprise or further comprise administration of an additional therapy.

The nucleic acids may be encoded on a viral vector. The viral vector may comprise an AAV vector. The virus may be packaged in HEK-293T cells.

The nucleic acid may comprise a lentivral vector backbone or a vector backbone that is based on the lentivirus. The viral vector may include or exclude a vector selected from Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). The nucleic acid may comprise an epHIV7 vector backbone. Other suitable viral vectors include, for example, pRSV-Rev, pMDLg/pRRE, psPAX2, pCMV delta R8.2, pMD2.G, pCMV-VSV-G, pCMV-dR8.2 dvpr, pCI-VSVG, pCPRDEnv, pLTR-RD114A, pLenti-III (Applied Biological Materials; cat #LV587); 87 pLentiCRISPR v.1 (Addgene; cat #52963); 88 p156RRLsinppt (Addgene; cat #42795); 89 pFUGW (Addgene; cat #14883); 90 pFUG (Addgene; cat #14882); 90 pHAGE (Addgene; cat #46793); 91 pHRsin (Addgene; cat #12265); 92 pLenti (AMP) (Addgene; cat #61422); 93 pLK0.1 (Addgene; cat #10878); 94 pLL3.7 m (Addgene; cat #89362); 95 Puro.cre (Addgene; cat #17408); 96 pRSIEG (Cellecta; SVSHU6EG-L); pLenti7.3 (Thermo Fisher Scientific; cat #V53406); pLenti (OriGene; cat #PS100109); pSF_Lenti (Sigma; cat #OGS269); pLV-GFPSpark (Sinobiological; cat #LVCV-01); pLVX (Takara; cat #632164); pALD-Lenti (Aldevron), pLenti (Vigene; cat #P100020), pLenti CMV (Addgene), and pLV. The vector backbone may be one that does not contain an antibiotic resistance gene. The vector backbone may be one that does not contain a beta-lactam resistance gene.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one.” but it is also consistent with the meaning of “one or more.” “at least one.” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1C: Higher doses of AAVs encoding for λN-DD E448Q+gRNA result in enhanced tumor growth suppression. 5×105 B16F10 melanoma cells were injected i.d. on day 0 followed by a single injection of AAVs i.t. on day 5 in combination with 100 μg mouse anti-PD-1 on days 5, 9, 13, and 17 i.p. (n=6). a) Tumor growth curves; mean+/−SEM, **p<0.01 by one-way ANOVA with Dunnett's post-test. b) Overall survival curves; **p<0.01 by log-rank (Mantel-Cox) tests between two groups. c) Individual mouse tumor growth curves.

FIG. 2A-2D: E448Q ADAR enzymes synergize with ICI to suppress tumor growth. 5×105 B16F10 melanoma cells were injected i.d. on day 0 followed by a single injection of AAVs i.t. on day 5 in combination with 100 μg mouse anti-PD-1 on days 5, 9, 13, and 17, and 21 i.p. (n=5-7). a,b) Tumor growth curves. c) Overall survival curves; *p<0.05. **p<0.01 by log-rank (Mantel-Cox) comparison to saline. d) Individual mouse tumor growth curves.

FIG. 3A-3F. Hyperactive ADAR synergizes with aPD1 to suppress tumor growth in immunologically cold B16F10 melanoma. B16F10 melanoma (500,000 cells, i.d.) was inoculated on day 0 followed by a single injection of AAVs (2×10¹⁰ gc, i.t) on day 5 in combination with αPD1 (100 μg. i.p.) every 4 days starting on day 5 (n=5-7). A) Average tumor growth curves, where points represent mean+/−SEM. B) Overall survival curve, where *p<0.05. **p<0.01 by log-rank (Mantel-Cox) comparison to PBS. C-F) Individual mouse tumor growth curves.

FIG. 4A-4F. Hyperactive λN-DD+gRNA synergizes with αPD1 to suppress tumor growth in immunologically cold B16F10 melanoma. B16F10 melanoma (500,000 cells, i.d.) was inoculated on day 0 followed by a single injection of AAVs (2×10¹⁰ gc. i.t) on day 5 in combination with αPD1 (100 μg. i.p.) every 4 days starting on day 5 (n=10-15). A) Average tumor growth curves, where points represent mean+/−SEM. B) Overall survival curve, where *p<0.05. ****p<0.0001 by log-rank (Mantel-Cox) comparison to GFP. C-F) Individual mouse tumor growth curves. Data pooled from 3 individual experiments.

FIG. 5A-5G. Higher doses of AAVs encoding for hyperactive λN-DD and gRNA suppress tumor growth to a greater extent. B16F10 melanoma (500,000 cells, i.d.) was inoculated on day 0 followed by a single injection of AAVs (0.25 to 2×10¹⁰ total gc at a 1:7 ratio of λN-DD: gRNA, i.t) on day 5 in combination with αPD1 (100 μg, i.p.) every 4 days starting on day 5 (n=6). Mice were euthanized when tumors reached 750 mm³ and/or based on humane end-point criteria. Surviving mice were euthanized at study endpoint on day 20. A) Average tumor growth curves, where points represent mean+/−SEM. B) Overall survival curve, where **p<0.01 by log-rank (Mantel-Cox) comparison to PBS. C-G) Individual mouse tumor growth curves.

FIG. 6A-6F. Treatment with hyperactive λN-DD and gRNA induces antigen-specific tumor-infiltrating lymphocytes (TILs). B16F10 melanoma (500,000 cells, i.d.) was inoculated on day 0 followed by a single injection of AAVs (2×10¹⁰ gc. i.t) on day 5 in combination with αPD1 (100 μg, i.p.) on days 5 and 9 (n=10). Lymphocytes within tumors were isolated on day 13, followed by flow cytometric analysis. A) Tumor measurements on day 13. B) CD45⁺CD3⁺CD8⁺ T cells per tumor weight (mg). C) CD3⁺CD8⁺ T cells as a percent of total CD45⁺ cells. D) TRP-2 pentamer⁺ cells as a percentage of CD3⁺CD8⁺ T cells. E-F). Representative flow cytometry plots in (D). Statistical significance indicated by *p<0.05 and **p<0.01 by unpaired t-test.

FIG. 7A-7F. Hyperactive λN-DD+gRNA synergizes with αPD1 to suppress tumor growth in immune excluded EMT6 breast cancer. 500,000 EMT6 breast cells were injected into the mammary fat pad of mice on day 0 followed by a single injection of AAVs (2×10¹⁰ gc. i.t). on day 5 in combination with αPD1 (100 μg, i.p.) on days 9, 13, 17, and 21 (n=11-13). A) Average tumor growth curves, where points represent mean+/−SEM. B) Overall survival curve, where *p<0.05 by log-rank (Mantel-Cox) comparison to GFP and CR indicates complete responders. C) On day 60, λN-DD EQ-treated survivors were rechallenged by injection of 500,000 EMT6 cells in the contralateral mammary fat pad. Naïve mice were also challenged as a control. The number of mice that developed palpable tumors is shown. D-F) Individual mouse tumor growth curves. Data pooled from two individual experiments.

### DETAILED DESCRIPTION

Current approaches to further enhance the immunogenicity of tumors include oncolytic viruses, gene therapy, and neoantigen vaccines. In 2015, the first oncolytic virus was approved by the U.S. Food and Drug Administration (FDA) for treatment of certain melanomas [3]. This therapy comprises a modified herpes simplex virus injected directly into cancerous lesions. The virus has been engineered to replicate specifically within tumor cells and to induce secretion of granulocyte-macrophage colony-stimulating factor to further promote immunogenicity. Other oncolytic viruses have been engineered to induce secretion of pro-inflammatory cytokines such as IL-7 and IL-12 and have been shown to enhance the effect of ICIs in preclinical combination studies [4, 5]. A major limitation of these viruses is their safety, specifically viral toxicity, off-target adverse effects, and potential unexpected consequences of genetic manipulation [6].

To enhance safety profiles for applications in gene delivery, adeno-associated viruses (AAVs) have been developed as non-pathogenic viruses that are non-replicating in the absence of helper viruses [7]. AAVs have been widely used in the literature for cancer gene therapies. Examples include their use to induce pro-inflammatory cytokines for enhanced immunogenicity, or to deliver tumor suppressor genes, anti-angiogenesis factors to inhibit vasculature formation and tumor growth, and antibodies to block signaling pathways [8]. However, the introduction of a single gene may not be sufficient to mount a robust immune response against the tumor.

As an alternative strategy, neoantigen vaccines have been designed to enhance the immunogenicity of new proteins arising from tumor mutations [9, 10]. Some neoantigens, such as those found in cancer driver genes, are shared across patients and cancer types. However, the vast majority of neoantigens that are MHC-restricted and immunogenic are patient-specific [11]. Therefore, much effort has been dedicated to identifying and vaccinating against these patient-specific neoantigens. Methods for identifying neoantigens include the direct identification of peptides from peptide-MHC complexes and the prediction of neoantigens from machine learning algorithms combined with next-generation sequencing [12]. While both of these methods have shown promise in neoantigen target identification, they are time and resource intensive and need to be individualized for each patient.

Once patient-specific neoantigens have been identified, there are two main strategies to vaccinate against them: adoptive cell therapy with dendritic cell vaccines or neoantigen-specific T cells and personalized vaccines against predicted neoantigens [9]. Personalized neoantigen vaccines can be composed of adjuvanted synthetic long peptides [15], mRNA [16, 17], or DNA encoding up to 20 patient-specific neoantigens. There are many ongoing clinical trials of neoantigen vaccines either alone or in combination with ICIs [10]. However, technical challenges in neoantigen identification and logistical challenges involving personalization remain. Furthermore, cancer cells could continue to evade the immune system through tumor escape mechanisms, and cells that do not contain the identified neoantigens may continue to proliferate in the process of immunoediting [19].

The methods described herein improves upon each of these by providing a safe and effective way to induce a multitude of neoantigens.

### I. ADMINISTRATION OF THERAPEUTIC COMPOSITIONS

The therapeutic cells of the disclosure may be administered by any route of administration. The cells may be administered intravenously, intramuscularly, subcutaneously, topically, orally, transdermally, intraperitoneally, intraorbitally, by implantation, by inhalation, intrathecally, intraventricularly, or intranasally. The composition, polypeptides, or viral particles may be administered by a route of administration that includes or excludes intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The cells can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### II. NUCLEIC ACIDS AND POLYPEPTIDES

### A. Nucleic Acids

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### 1. Vector

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

### 2. Regulatory Elements

Eukaryotic expression cassettes included in the vectors particularly contain (in a 5′-to-3′ direction) a eukaryotic transcriptional promoter operably linked to a protein-coding sequence, splice signals including intervening sequences, and a transcriptional termination/polyadenylation sequence.

a. Promoter/Enhancers

A “promoter” is a control sequence that is a region of a nucleic acid sequence at which initiation and rate of transcription are controlled. In aspects of the disclosure, the promoter or enhancer may include a promoter or enhancer from the promoter region and/or gene of Table 1. It may contain genetic elements at which regulatory proteins and molecules may bind, such as RNA polymerase and other transcription factors, to initiate the specific transcription a nucleic acid sequence. The phrases “operatively positioned.” “operatively linked.” “under control,” and “under transcriptional control” mean that a promoter is in a correct functional location and/or orientation in relation to a nucleic acid sequence to control transcriptional initiation and/or expression of that sequence.

The promoter region of the disclosure may be an endogenous promoter region. An endogenous promoter region refers to the situation in which the promoter region is in it's endogenous genomic setting, such that the sequences upstream of the promoter (i.e. 5′ region) are the substantially the same as those that are in the wild-type cell. Substantially the same could refer to a region that is at least 80, 85, 90, 95, 96, 97, 98, or 99% identical to the upstream region of the wild-type. An endogenous promoter region also refers to a situation in which the promoter is in the same genomic location as the wild-type promoter. In some aspects, the endogenous promoter refers to a promoter in a cell this is unmodified or that is or is at least 70, 75, 80, 85, 90, 95, 96, 97, 98, or 99% identical to the wild-type promoter.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

b. Protease Cleavage Sites/Self-Cleaving Peptides and Internal Ribosome Binding Sites

Suitable protease cleavages sites and self-cleaving peptides are known to the skilled person (scc, e.g., in Ryan et al., 1997; Scymczak et al., 2004). Examples of protease cleavage sites are the cleavage sites of potyvirus NIa proteases (e.g. tobacco etch virus protease), potyvirus HC proteases, potyvirus P1 (P35) proteases, byovirus Nla proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PY\IF (parsnip yellow fleck virus) 3C-like protease, thrombin, factor Xa and enterokinase. Due to its high cleavage stringency, TEV (tobacco etch virus) protease cleavage sites may be used.

Exemplary self-cleaving peptides (also called “cis-acting hydrolytic elements”, CHYSEL; see deFelipe (2002) are derived from potyvirus and cardiovirus 2A peptides. Particular self-cleaving peptides may be selected from 2A peptides derived from FMDV (foot-and-mouth disease virus), equine rhinitis A virus, Thoseà asigna virus and porcine teschovirus.

A specific initiation signal also may be used for efficient translation of coding sequences in a polycistronic message. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain aspects, the use of internal ribosome entry sites (IRES) elements are used to create multigene, or polycistronic, messages. IRES elements are able to bypass the ribosome scanning model of 5□ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs may comprise at least one termination signal. A “termination signal” or “terminator” is comprised of the DNA sequences involved in specific termination of an RNA transcript by an RNA polymerase. Thus, in certain aspects a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this poly A tail appear to more stable and are translated more efficiently. Thus, in other aspects involving eukaryotes, the terminator comprises a signal for the cleavage of the RNA, and the terminator signal promotes polyadenylation of the message. The terminator and/or polyadenylation site elements can serve to enhance message levels and to minimize read through from the cassette into other sequences.

Terminators contemplated include any known terminator of transcription described herein or known to one of ordinary skill in the art, including but not limited to, for example, the termination sequences of genes, such as for example the bovine growth hormone terminator or viral termination sequences, such as for example the SV40 terminator. In certain aspects, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice, and any such sequence may be employed. Exemplary aspects include the SV40 polyadenylation signal or the bovine growth hormone polyadenylation signal, convenient and known to function well in various target cells. Polyadenylation may increase the stability of the transcript or may facilitate cytoplasmic transport.

### 3. Vector Delivery

Genetic modification or introduction of transgene nucleic acids into cells of the disclosure may use any suitable methods for nucleic acid delivery for transformation of a cell, as described herein or as would be known to one of ordinary skill in the art. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Such methods include, but are not limited to, direct delivery of DNA such as by ex vivo transfection (Wilson et al., 1989, Nabel et al. 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirullch et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al. (2001) Molecular Cloning: A Laboratory Manual, Cold Spring Harbor Laboratory, New York). One illustrative, but non-limiting method for the introduction of a polynucleotide into a host cell is calcium phosphate transfection.

Biological methods for introducing a polynucleotide of interest into a host cell can include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (scc, e.g. U.S. Pat. Nos. 5,350,674 and 5,585,362, and the like).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An illustrative colloidal system for use as a delivery vehicle in vitro and in vivo is a liposome (e.g., an artificial membrane vesicle).

### 4. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5×sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### 5. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### 6. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred aspect, the nucleic acid molecules are oligonucleotides. In a more preferred aspect, the oligonucleotides are from highly variable regions of the heavy and light chains of the antibody of interest. In an even more preferred aspect, the oligonucleotides encode all or part of one or more of the CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### B. Polypeptides

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS: 1-11. In specific aspects, the peptide or polypeptide is or is based on a human sequence. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS: 1-11 (or any derivable range therein) and may be a substitution with any amino acid or may be a substitution with a alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-11.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-11 and have or have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOS: 1-11.

In some aspects, the protein, polypeptide, or nucleic acid may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids or nucleic acids of SEQ ID NOS: 1-11.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS: 1-11 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS: 1-11.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 of any of SEQ ID NOS: 1-11 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids or nucleotides of any of SEQ ID NOS: 1-11.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### III. ADDITIONAL THERAPIES

### A. Immunotherapy

In some aspects, the methods comprise administration of an additional therapy. In some aspects, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Checkpoint Inhibitors and Combination Treatment

Aspects of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7. TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 4. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### B. Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### C. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

### D. Surgery

In some aspects, the additional therapy comprises surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months (or any range derivable therein). These treatments may be of varying dosages as well.

### IV. TREATMENT OF DISEASE

Methods may be employed with respect to individuals who have tested positive for such disorders or who are deemed to be at risk for developing such a condition or related condition. In some aspects, the compositions and methods described herein are used to treat a cancer. In some aspects, the cancer comprises a solid tumor.

Certain aspects of the disclosure relate to the treatment of. The cancer to be treated may be any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. In some aspects, the cancer to be treated is from one of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, glioblastoma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome.

### V. EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1: Methods of Inducing Neoantigens in Tumors

The inventors hypothesized that the induction of a multitude of neoantigens in tumor cells, through hundreds of thousands of RNA editing events, would render tumors more recognizable by the patient's own T cells and that this could serve as an off-the-shelf therapeutic and could enhance the efficacy of ICIs. They tested this hypothesis through the local delivery of hyperactive adenosine-to-inosine (A-to-I) RNA editing enzymes.

### A. Experimental

Adenosine deaminases that act on RNA (ADARs) are enzymes in multicellular metazoans that selectively deaminate adenosine into inosine, which is subsequently read as guanosine during translation. Therefore, ADARs can change protein sequences on the codon level and have been recently explored as therapeutic molecular tools, for example in the context of cystic fibrosis and other genetic disorders [20, 21]. It was found that a hyperactive ADAR2 (E488Q) causes high off-target editing [25]. This mutant produced hundreds of thousands of off-target A-to-I edits across the transcriptome [25]. Off-target edits are problematic for site-directed RNA editing in a therapeutic context because they could cause unintended toxicity. It was hypothesized that these off-target edits could prove advantageous for immunotherapies by encoding neoantigens and enhancing ICIs in cold tumors. Therefore, the inventors sought to determine whether administration of the hyperactive ADAR E488Q-based enzyme can enhance the efficacy of cancer immunotherapy.

### B. Methods

AAVs are approved for gene therapies and are leading candidates to deliver RNA editing components in vivo [27, 28]. Therefore, the inventors used AAVs encoding for wild type (WT) ADAR2, ADAR2 E488Q, WT λN-DD, λN-DD E448Q, and a generic gRNA (any small gRNA delivered in combination with λN-DD E448Q that produces widespread off-target edits in this system; [25]). Here, the inventors used gRNA complementary to the region around cGFP codon 58 without any BoxB hairpins. Since BoxB hairpins are only necessary for specific editing, and since they inhibit packaging in AAVs, the inventors used a gRNA that lacks a BoxB region. They also used separate AAVs encoding for λN-DD and gRNA. The AAV serotype used was DJ, which is derived from eight different serotypes including 2, 8, and 9 for improved transfection efficiency across a broad range of cell types [29].

As proof-of-concept of this approach, the inventors tested it in a highly aggressive, immunologically cold model of murine melanoma (B16F10) [30]. This model has moderate mutational burden and low immunogenicity characterized by limited T cell infiltration and lack of response to ICI monotherapies [30]. Therefore, it is often employed in preclinical evaluations of novel cancer immunotherapies. In this syngeneic model, 5×10⁵ melanoma cells derived from C57Bl/6 mice are injected intradermally (i.d.) into recipient healthy C57Bl/6 mice to inoculate the tumor. Mice received a single intratumoral (i.t.) injection of AAVs 5 days post-inoculation once tumors reached ˜15 mm³. Anti-mouse PD-1 (Clone: 29F.1A12, 100 μg/mouse) was dosed intraperitoneally (i.p.) every 4 days starting on day 5.

### C. Results

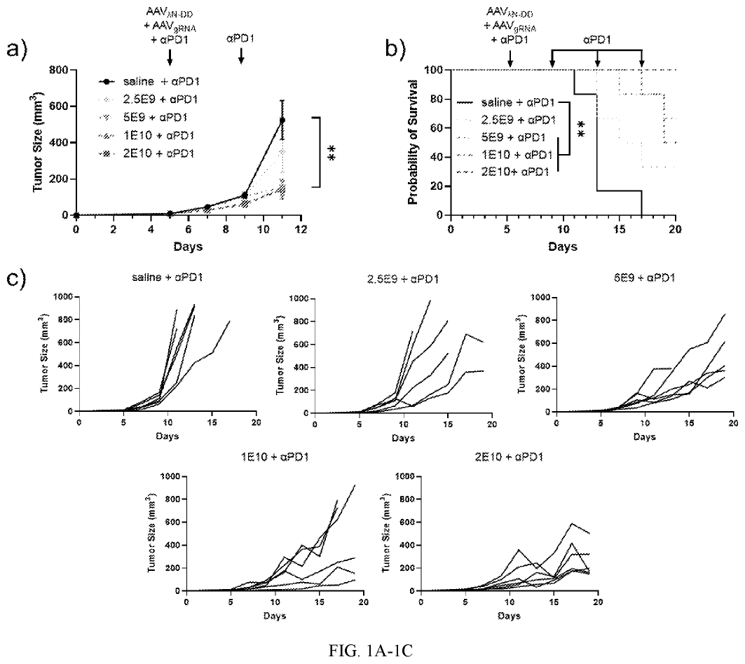

The inventors first conducted a dose-escalation study of λN-DD E448Q+gRNA combined with anti-PD-1 in the B16F10 mouse model of melanoma with AAV doses ranging from 2.5×10⁹ genome copies/mouse (2.5E9) to 2×10¹⁰ genome copies/mouse (2E10; FIG. 1). The ratio of AAVs encoding for λN-DD E448Q to AAVs encoding for gRNA remained constant at 1:7. For example, the 2E10 dose contained 2.5E9 λN-DD E488Q and 1.75E10 gRNA. There was a significant reduction in tumor growth for the 5E9, 1E10, and 2E10 doses compared to saline 11 days after tumor inoculation (FIG. 1a). The median survival of mice in the saline group was 13 days with 0% survival by day 17, and overall survival was enhanced with increasing doses of AAVs. Importantly, no mortality was observed (n=6) in the 2E10 group by day 20 upon study completion, suggesting that higher doses are more efficacious (FIG. 1b). Individual growth curves validate these findings, showing that mice receiving the highest dose (2E10) have consistently inhibited tumor growth (FIG. 1c).

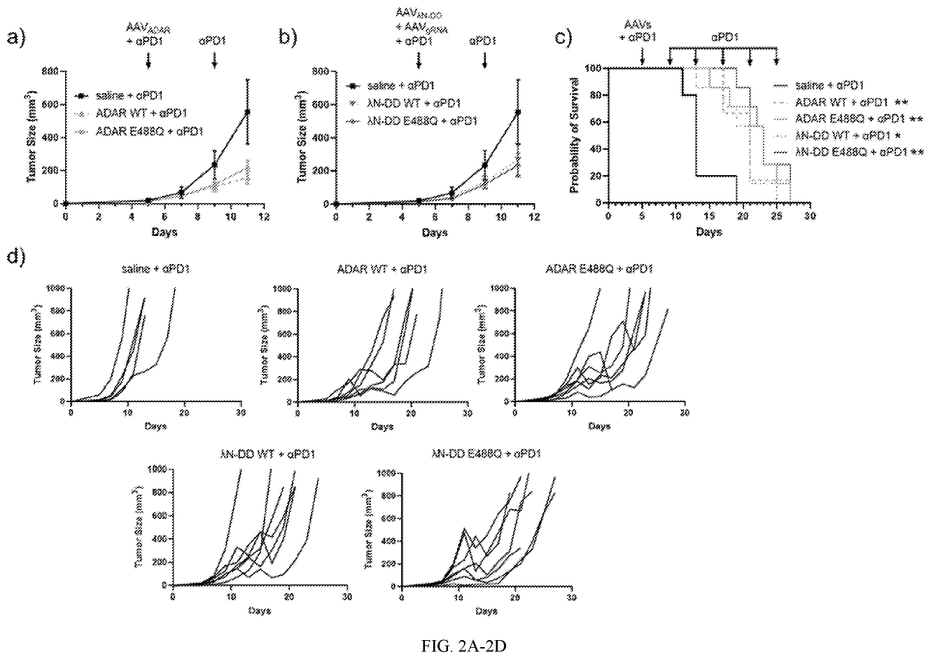

The inventors next compared the efficacy of WT ADAR enzymes and WT λN-DD+gRNA and their E448Q counterparts to anti-PD1 as a monotherapy (FIG. 2). Neither ADAR E488Q nor λN-DD E488Q as a monotherapy resulted in a significant reduction in tumor growth compared to anti-PD1 monotherapy (FIG. 2a,b). However, the synergistic effect of the combination therapy resulted in significantly prolonged survival for both WT and E488Q enzymes (FIG. 2c). The median survival for the group receiving saline with anti-PD1 was 13 days, while that for mice receiving WT or E488Q enzymes was 21 or 23 days, respectively, regardless of whether the ADAR2 or the λN-DD system was used (FIG. 2c). The individual growth curves show that the E488Q enzymes suppressed tumor growth to the greatest extent. Therefore, these RNA editing enzyme systems may enhance the efficacy of ICI in immunologically cold cancers.

### Example 2: Methods for Treating Cancer with Hyperactive ADAR Enzymes

### A. Methods

### 1. Mice and Cancer Cell Lines

Female C57BL/6 or Balb/c mice (aged 7-9 weeks) were purchased from Charles River Laboratory. B16F10 murine melanoma and EMT6 murine mammary carcinoma were purchased from ATCC and cultured according to instructions. All animal experiments performed in this work were approved by the Institutional Animal Care and Use Committee of the University of Chicago.

### 2. AAVs

Adeno-associated viruses, serotype DJ, containing plasmids encoding for the wild type (WT) or hyperactive (EQ) ADAR enzymes, λN-DD WT or EQ. gRNA (complementary to the region around eGFP codon 58 without any BoxB hairpins), and eGFP were produced by core facilities or ordered commercially and verified in house.

### 3. Tumor Survival Experiments

Tumors were inoculated with 500,000 cells in 30 μL of sterile PBS injected into the dermis (B16F10) or mammary fat pad (EMT6), in C57BL/6 and Balb/c respectively. Starting 5 days post-inoculation and every 2 days thereafter tumor dimensions were measured with digital calipers, and volume was calculated as length×width×height×(π/6). On day 5 post-inoculation, mice were administered intratumoral injections of AAVs. Mice were administered anti-mouse PD-1 (Clone: 29F.1A12, 100 μg/mouse) was intraperitoneally (i.p.) every 4 days starting on day 5 (B16F10) or day 9 (EMT6) until euthanasia criteria were met (B16F10) or for a total of 4 doses (EMT6). Mice were euthanized when the tumor volume exceeded 750 mm³ and/or based on humane end-point criteria.

### 4. T Cell Infiltrates in B16F10 Melanoma

On day 13, mice were euthanized and tumors were harvested. Tumors were digested with collagenase D, collagenase IV, and DNAse I for 30 min at 37° C. Single-cell suspensions were prepared using a 70 μm cell strainer, and tumor-infiltrating lymphocytes (TIL) were isolated with a density gradient using Ficoll-Paque PREMIUM 1.084. The mononuclear cells were plated and stained with CD45 (clone 30-F11, BioLegend), CD38 (clone 145-2C11, BD Biosciences), CD8α (clone 53-6.7, BioLegend), CD4 (clone RM4-5, BioLegend), and TRP-2 MHC I pentamer (ProImmune). Samples were run on a BD LSR Fortessa, and data was analyzed using FlowJo software.

### 5. Statistics

Statistical analysis was performed using GraphPad Prism. For direct comparisons of two groups, unpaired, two-tailed t-tests were used. For survival analyses, pairwise log-rank (Mantel-Cox) tests were used.

### B. Results

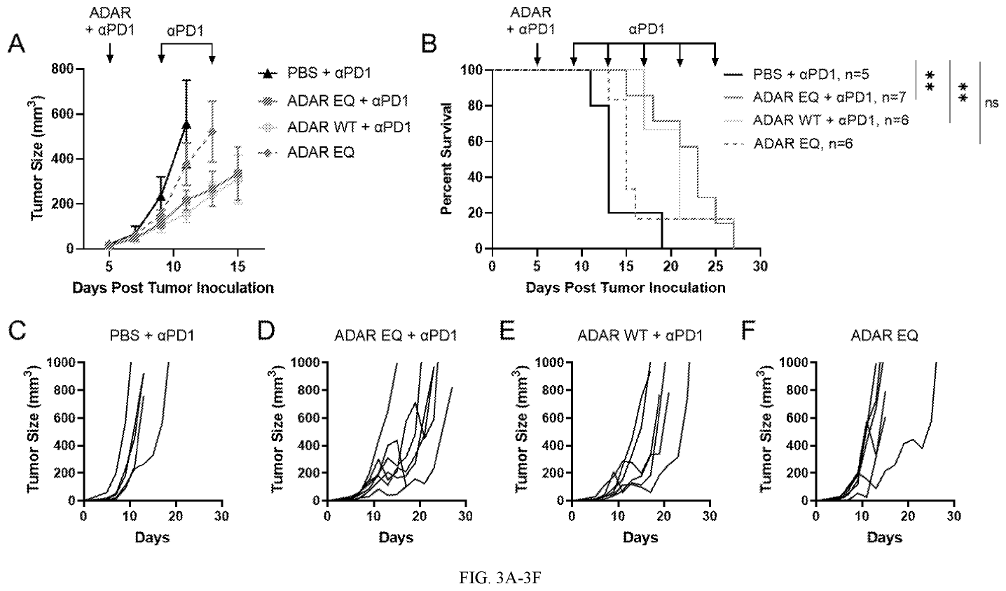

To test the efficacy of this approach, the inventors used a highly aggressive, immunologically cold model of murine melanoma (B16F10) with moderate existing mutational burden and low immunogenicity. B16F10 is characterized by limited T cell infiltration and lack of response to ICI monotherapies, making it a highly challenging preclinical model. They first tested whether a single intratumoral injection of AAVs encoding native (WT) or hyperactive (EQ) ADAR could synergize with αPD1 ICI to suppress tumor growth (FIG. 3). In combination with αPD1, the inventors found that both ADAR WT and ADAR EQ slow tumor growth, resulting in a significant survival benefit. In the absence of αPD1, there are no differences in survival rates between ADAR EQ-treated and control mice.

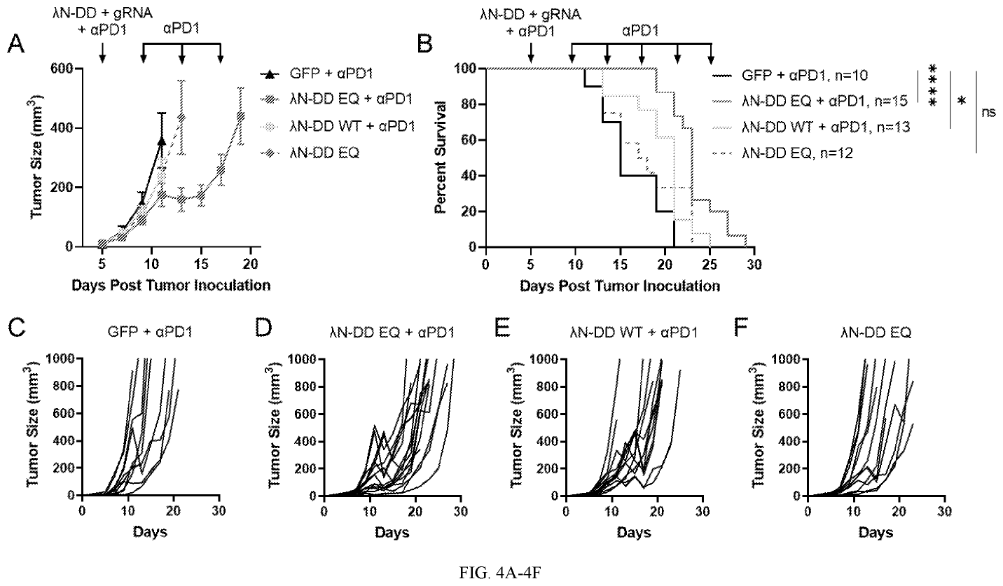

The inventors also wanted to test the engineered λN-DD system in combination with gRNA in this model (FIG. 4). Similar to ADAR enzymes, they observed a suppression in tumor growth and prolonged survival for mice treated with λN-DD WT and a more pronounced effect for λN-DD EQ. In these experiments, the inventors use a control that has AAVs encoding for eGFP and show that neither the delivery vehicle nor the expression of a foreign protein has a significant effect on tumor growth.

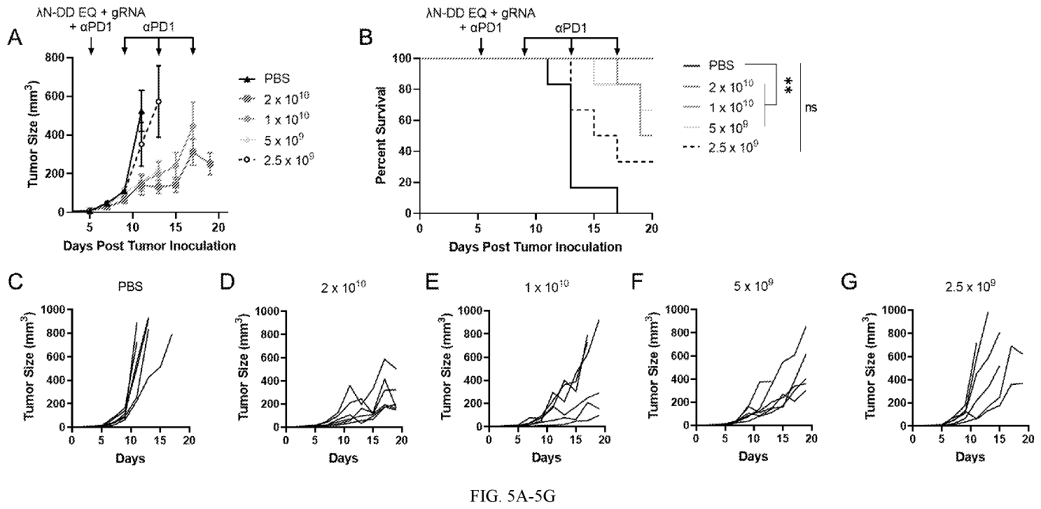

Next, the inventors performed a dose escalation study for the λN-DD system, increasing the total number of total genome copies (gc) per mouse from 2.5×10⁹ to 2×10¹⁰ (FIG. 5). The dose and ratio of AAVs encoding λN-DD EQ to gRNA (1:7) were determined based on prior in vitro studies and pilot studies in mice (data not shown). After a total of 20 days, 0/6 mice receiving αPD1 alone and 6/6 mice receiving the highest dose of λN-DD+gRNA in combination with αPD1 survived.

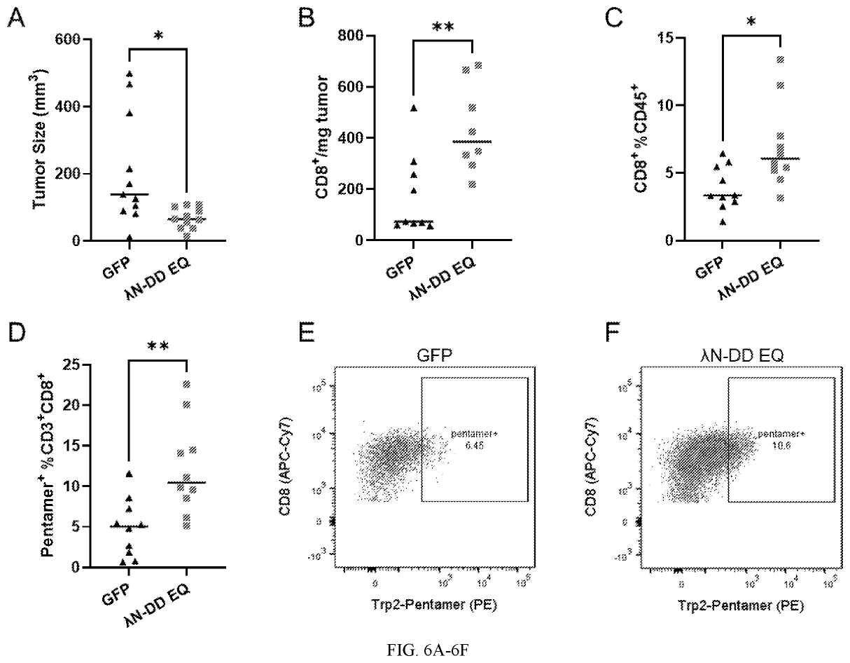

To probe the immunological response to λN-DD EQ and gRNA, the inventors harvested tumors from mice and performed a flow cytometric analysis of tumor infiltrating lymphocytes (TILs, FIG. 6). Tumors treated with AAVs for λN-DD EQ and gRNA were significantly smaller than those treated with AAVs encoding GFP (FIG. 6A). The λN-DD EQ and gRNA-treated tumors had more cytotoxic CD8+ T cells, both per weight of tumor and per total immune cells in the tumor (FIG. 6 B,C). The inventors also used a MHC class I pentamer for TRP-2 to determine the number of T cells specific to this immunodominant, native B16F10 antigen. They observed more TRP-2-specific CD8+ T cells in terms of counts and percentage of total CD8+ T cells in λN-DD EQ and gRNA-treated tumors (FIG. 6 D,E).

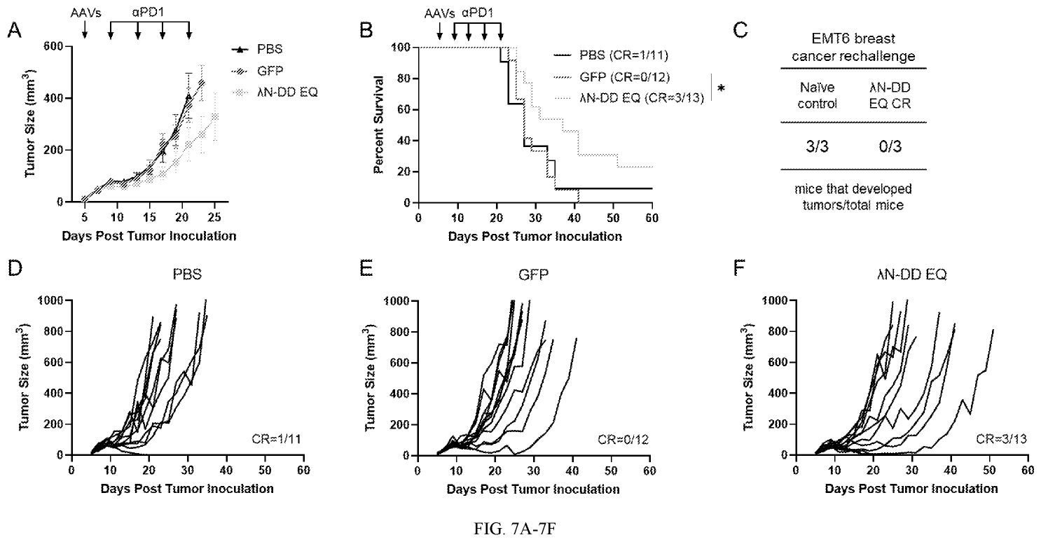

Finally, the inventors tested the λN-DD system in an immune-excluded model of breast cancer (EMT6, FIG. 7). EMT6 has a relatively low mutational burden and is partially responsive to αPD1. The inventors followed a similar timeline as B16F10 but started dosing αPD1 9 days post-inoculation, and gave a maximum of 4 doses per animal. The inventors found that λN-DD EQ and gRNA slows tumor growth and results in complete tumor regression in 3 of 13 animals. Moreover, animals that completely responded to treatment did not grow palpable tumors upon EMT6 challenge in the contralateral mammary fat pad 60 days post-inoculation, indicative of immune memory. The use of this model demonstrates the generalizability of our approach to multiple cancer types.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. The references cited in the disclosure, to the extent that they provide exemplary procedural or other details supplementary to those set forth herein, are specifically incorporated herein by reference.

The references cited herein (including the following references), to the extent that they provide exemplary procedural or other details supplementary to those set forth herein, are specifically incorporated herein by reference.
[1] Weigmann, Katrin. “Releasing the brakes to fight cancer: The recent discovery of checkpoints has boosted the field of cancer immunotherapy.” EMBO reports 17, no. 9 (2016): 1257-1260.
[2] Postow, Michael A., Margaret K. Callahan, and Jedd D. Wolchok. “Immune checkpoint blockade in cancer therapy.” Journal of clinical oncology 33, no. 17 (2015): 1974.
[3] Pol, Jonathan, Guido Kroemer, and Lorenzo Galluzzi. “First oncolytic virus approved for melanoma immunotherapy.” (2016): e1115641.
[4] Nakao, Shinsuke, Yukinori Arai, Mamoru Tasaki, Midori Yamashita, Ryuji Murakami, Tatsuya Kawase, Nobuaki Amino et al. “Intratumoral expression of IL-7 and IL-12 using an oncolytic virus increases systemic sensitivity to immune checkpoint blockade.” Science translational medicine 12, no. 526 (2020).
[5] Zhang, Wei, Clark C. Chen, and Jianfang Ning. “Combining oncolytic virus with FDA approved pharmacological agents for cancer therapy.” Expert opinion on biological therapy 21, no. 2 (2021): 183-189.
[6] Lawler, Sean E., Maria-Carmela Speranza, Choi-Fong Cho, and E. Antonio Chiocca. “Oncolytic viruses in cancer treatment: a review.” JAMA oncology 3, no. 6 (2017): 841-849.
[7] Wang, Dan, Phillip WL Tai, and Guangping Gao. “Adeno-associated virus vector as a platform for gene therapy delivery.” Nature reviews Drug discovery 18, no. 5 (2019): 358-378.
[8] Santiago-Ortiz, Jorge L., and David V. Schaffer. “Adeno-associated virus (AAV) vectors in cancer gene therapy.” Journal of Controlled Release 240 (2016): 287-301.
[9] Blass, Eryn, and Patrick A. Ott. “Advances in the development of personalized neoantigen-based therapeutic cancer vaccines.” Nature Reviews Clinical Oncology 18, no. 4 (2021): 215-229.
[10] Yarchoan, Mark, Burles A. Johnson, Eric R. Lutz, Daniel A. Laheru, and Elizabeth M. Jaffee. “Targeting neoantigens to augment antitumour immunity.” Nature Reviews Cancer 17, no. 4 (2017): 209-222.
[11] Pearlman, Alexander H., Michael S. Hwang. Maximilian F. Konig, Emily Han-Chung Hsiue, Jacqueline Douglass, Sarah R. DiNapoli, Brian J. Mog et al. “Targeting public neoantigens for cancer immunotherapy.” Nature Cancer 2, no. 5 (2021): 487-497.
[12] Chen, Ina, Michael Y. Chen, S. Peter Goedegebuure, and William E. Gillanders. “Challenges targeting cancer neoantigens in 2021: a systematic literature review.” Expert Review of Vaccines just-accepted (2021).
[13] Saxena, Mansi, and Nina Bhardwaj. “Re-emergence of dendritic cell vaccines for cancer treatment.” Trends in cancer 4, no. 2 (2018): 119-137.
[14] Yamamoto, Tori N., Rigel J. Kishton, and Nicholas P. Restifo. “Developing neoantigen-targeted T cell-based treatments for solid tumors.” Nature medicine 25, no. 10 (2019): 1488-1499.
[15] Cohen, Roger B., Melissa Johnson, Przemyslaw Twardowski, Mark Stein, Ulka Vaishampayan, Maura Gillison, Lisa McNeil et al. “Broad immunogenicity from GEN-009, a neoantigen vaccine using ATLAS™, an autologous immune assay, to identify immunogenic and inhibitory tumor neoantigens.” J. Immunother. Cancer 7, no. Suppl. 1 (2019): P420.
[16] Cafri, Gal, Jared J. Gartner, Tal Zaks, Kristen Hopson, Noam Levin, Biman C. Paria, Maria R. Parkhurst et al. “mRNA vaccine-induced neoantigen-specific T cell immunity in patients with gastrointestinal cancer.” The Journal of clinical investigation 130, no. 11 (2020).
[17] Sahin, Ugur, Evelyna Derhovanessian, Matthias Miller, Björn-Philipp Kloke, Petra Simon, Martin Löwer, Valesca Bukur et al. “Personalized RNA mutanome vaccines mobilize poly-specific therapeutic immunity against cancer.” Nature 547, no. 7662 (2017): 222-226.
[18] Krauss, Jurgen, Angela Krackhardt, Elke Jaeger, Anja Williams, Reza Rafiyan, Hedda Wold, Lisa Gerner et al. “Preliminary safety, efficacy and immunogenicity results from a phase 1/2a study (DIRECT-01) of cancer neoantigen DNA vaccine VB10. NEO in patients with locally advanced or metastatic solid tumors.” (2019).
[19] Fritsch, Edward F., Ute E. Burkhardt, Nir Hacohen, and Catherine J. Wu. “Personal neoantigen cancer vaccines: a road not fully paved.” Cancer immunology research 8, no. 12 (2020): 1465.
[20] Montiel-Gonzalez, Maria Fernanda, Juan Felipe Diaz Quiroz, and Joshua JC Rosenthal. “Current strategies for site-directed RNA editing using ADARs.” Methods 156 (2019): 16-24.
[21] Montiel-Gonzalez, Maria Fernanda, Isabel Vallecillo-Viejo, Guillermo A. Yudowski, and Joshua JC Rosenthal. “Correction of mutations within the cystic fibrosis transmembrane conductance regulator by site-directed RNA editing.” Proceedings of the National Academy of Sciences 110, no. 45 (2013): 18285-18290.
[22] Kuttan, Ashani, and Brenda L. Bass. “Mechanistic insights into editing-site specificity of ADARs.” Proceedings of the National Academy of Sciences 109, no. 48 (2012): E3295-E3304.
[23] Phelps, Kelly J., Kiet Tran, Tristan Eifler, Anna I. Erickson, Andrew J. Fisher, and Peter A. Beal. “Recognition of duplex RNA by the deaminase domain of the RNA editing enzyme ADAR2.” Nucleic acids research 43, no. 2 (2015): 1123-1132.
[24] Montiel-González, Maria Fernanda, Isabel C. Vallecillo-Viejo, and Joshua JC Rosenthal. “An efficient system for selectively altering genetic information within mRNAs.” Nucleic acids research 44, no. 21 (2016): e157-157.
[25] Vallecillo-Viejo, Isabel C., Noa Liscovitch-Brauer, Maria Fernanda Montiel-Gonzalez, Eli Eisenberg, and Joshua JC Rosenthal. “Abundant off-target edits from site-directed RNA editing can be reduced by nuclear localization of the editing enzyme.” RNA biology 15, no. 1 (2018): 104-114.
[26] Kuzmin, Dmitry A., Maria V. Shutova, Natalie R. Johnston, Owen P. Smith, Vasily V. Fedorin, Yury S. Kukushkin, Johannes CM van der Loo, and Elaine C. Johnstone. “The clinical landscape for AAV gene therapies.” Nature reviews. Drug Discovery (2021).
[27] Sinnamon, John R., Susan Y. Kim, Jenna R. Fisk, Zhen Song, Hiroyuki Nakai, Sophia Jeng, Shannon K. McWeeney, and Gail Mandel. “In vivo repair of a protein underlying a neurological disorder by programmable RNA editing.” Cell reports 32, no. 2 (2020): 107878.
[28] Sinnamon, John R., Susan Y. Kim, Glen M. Corson, Zhen Song, Hiroyuki Nakai, John P. Adelman, and Gail Mandel. “Site-directed RNA repair of endogenous Mecp2 RNA in neurons.” Proceedings of the National Academy of Sciences 114, no. 44 (2017): E9395-E9402.
[29] Grimm, Dirk, Joyce S. Lee, Lora Wang, Tushar Desai, Bassel Akache, Theresa A. Storm, and Mark A. Kay. “In vitro and in vivo gene therapy vector evolution via multispecies interbreeding and retargeting of adeno-associated viruses.” Journal of virology 82, no. 12 (2008): 5887-5911.
[30] Yu, Jong W., Sabyasachi Bhattacharya, Niranjan Yanamandra, David Kilian, Hong Shi, Sapna Yadavilli, Yuliya Katlinskaya et al. “Tumor-immune profiling of murine syngeneic tumor models as a framework to guide mechanistic studies and predict therapy response in distinct tumor microenvironments.” PloS one 13, no. 11 (2018): e0206223.
[31] Xue, Diyuan, Eric Hsu, Yang-Xin Fu, and Hua Peng. “Next-generation cytokines for cancer immunotherapy.” Antibody therapeutics 4, no. 2 (2021): 123-133.
[32] Berraondo, Pedro, Miguel F. Sanmamed, María C. Ochoa, Iñaki Etxeberria, Maria A. Aznar, José Luis Pérez-Gracia, María E. Rodríguez-Ruiz, Mariano Ponz-Sarvise, Eduardo Castañón, and Ignacio Melero. “Cytokines in clinical cancer immunotherapy.” British journal of cancer 120, no. 1 (2019): 6-15.
[33] Ishihara, Jun, Kazuto Fukunaga, Ako Ishihara, Hans M. Larsson, Lambert Potin, Peyman Hosseinchi, Gabriele Galliverti, Melody A. Swartz, and Jeffrey A. Hubbell. “Matrix-binding checkpoint immunotherapies enhance antitumor efficacy and reduce adverse events.” Science translational medicine 9, no. 415 (2017).
[34] Ishihara, Jun, Ako Ishihara, Koichi Sasaki, Steve Seung-Young Lee, John-Michael Williford, Mariko Yasui, Hiroyuki Abe et al. “Targeted antibody and cytokine cancer immunotherapies through collagen affinity.” Science translational medicine 11, no. 487 (2019).
[35] Mansurov, Aslan, Jun Ishihara, Peyman Hosseinchi, Lambert Potin, Tiffany M. Marchell, Ako Ishihara, John-Michael Williford et al. “Collagen-binding IL-12 enhances tumour inflammation and drives the complete remission of established immunologically cold mouse tumours.” Nature biomedical engineering 4, no. 5 (2020): 531-543.
[36] Vonderheide, Robert H. “CD40 agonist antibodies in cancer immunotherapy.” Annual review of medicine 71 (2020): 47-58.
[37] Ribas, Antoni, and Jedd D. Wolchok. “Cancer immunotherapy using checkpoint blockade.” Science 359, no. 6382 (2018): 1350-1355.
[38] Vogel, Paul, Matin Moschref, Qin Li, Tobias Merkle, Karthika D. Selvasaravanan, Jin Billy Li, and Thorsten Stafforst. “Efficient and precise editing of endogenous transcripts with SNAP-tagged ADARs.” Nature methods 15, no. 7 (2018): 535-538.
[39] Azad, Md TA, S. Bhakta, and T. Tsukahara. “Site-directed RNA editing by adenosine deaminase acting on RNA for correction of the genetic code in gene therapy.” Gene therapy 24, no. 12 (2017): 779-786.
[40] Cox, David B T, Jonathan S. Gootenberg, Omar O. Abudayyeh, Brian Franklin, Max J. Kellner, Julia Joung, and Feng Zhang. “RNA editing with CRISPR-Cas13.” Science 358, no. 6366 (2017): 1019-1027.

What is claimed is:

1. A method for treating cancer in a subject comprising administering a composition comprising a polypeptide comprising an adenosine deaminase RNA-specific binding protein (ADAR) or fragment thereof or a nucleic acid encoding a polypeptide comprising an adenosine deaminase RNA-specific binding protein (ADAR) or fragment thereof, wherein the composition is administered in combination with an immunotherapy.

2. The method of claim 1, wherein the ADAR or fragment comprises a hyperactive ADAR or a hyperactive ADAR fragment.

3. The method of claim 1 or 2, wherein the polypeptide further comprises a lambda phage N protein (λN).

4. The method of claim 3, wherein the λN comprises SEQ ID NO:2 or an amino acid sequence with at least 70% sequence identity to SEQ ID NO:2.

5. The method of any one of claims 1-4, wherein the polypeptide further comprises a SNAP tag.

6. The method of any one of claims 1-5, wherein the method further comprises administration of an antisense RNA or a DNA encoding an antisense RNA.

7. The method of any one of claims 2-6, wherein the hyperactive ADAR or fragment has an A-to-G whole genome editing index of greater than 0.35 or an A-to-G Alu element editing index of greater than 6.

8. The method of any one of claims 1-7, wherein the method further comprises administration of viral particle(s) encoding an antisense RNA.

9. The method of any one of claims 6-8, wherein the antisense RNA excludes a hairpin.

10. The method of any one of claims 6-9, wherein the antisense RNA excludes a BoxB hairpin.

11. The method of any one of claims 6-11, wherein the antisense RNA is not complimentary to a genomic sequence.

12. The method of any one of claims 6-8, wherein the antisense RNA comprises at least one hairpin.

13. The method of claim 12, wherein the at least one hairpin comprises a BoxB hairpin.

14. The method of claim 12 or 13, wherein the antisense RNA comprises at least two hairpins.

15. The method of any one of claims 12-14, wherein at least one hairpin comprises a nucleotide sequence of GGCCCTGAAAAAGGGCC (SEQ ID NO:9).

16. The method of any one of claims 12-15, wherein the antisense RNA comprises a nucleotide sequence that is complimentary to a cancer neoantigen.

17. The method of claim 16, wherein the neoantigen is selected from Braf p.Val600Glu; KRAS p.Gly12Asp; KRAS p.Gly12Val; TP53 p.Arg175His; KRAS p.Gly12Asp; KRAS p.Gly12Val; HRAS//KRAS/NRAS pGln61Arg; KRAS p.Gly12Val; BRAF p.Val600Glu; and KRAS p.Gly12Asp.

18. The method of any one of claims 6-17, wherein the antisense RNA comprises less than 90 nucleotides.

19. The method of any one of claims 6-18, wherein the antisense RNA comprises less than 60 nucleotides.

20. The method of any one of claims 1-17, wherein the immunotherapy comprises an immune checkpoint inhibitor (ICI), an immune agonist antibody, or nucleic acids encoding for an immune checkpoint inhibitor or an immune agonist antibody.

21. The method of claim 20, wherein the ICI or immune agonist antibody comprises an anti-CD40 agonistic antibody, anti-PD1 blocking antibody, anti-PDL1 blocking antibody, anti-CTLA4 blocking antibody, or combinations thereof.

22. The method of claim 21, wherein the ICI comprises one or more of Ipilimumab, Tremelimumab, Nivolumab, Pembrolizumab, Atezolizumab, Avelumab, Durvalumab, Spartalizumab, and Cemiplimab.

23. The method of any one of claims 1-22, wherein the immunotherapy comprises pro-inflammatory molecules or nucleic acids encoding for pro-inflammatory molecules.

24. The method of claim 23, wherein the pro-inflammatory molecule comprises IL-2, IL-12, IL-15, or combinations thereof.

25. The method of any one of claims 1-24, wherein the polypeptide and/or antisense RNA is administered before the immunotherapy or at approximately the same time as the immunotherapy.

26. The method of any one of claims 20-25, wherein the ICI therapy comprises monotherapy or combination therapy.

27. The method of claim 26, wherein the ICI therapy comprises an anti-PD1 antibody.

28. The method of any one of claims 1-27, wherein the ADAR or fragment comprises an E448Q substitution.

29. The method of any one of claims 1-28, wherein the ADAR or fragment comprises ADAR2 or fragment thereof.

30. The method of any one of claims 1-29, wherein the ADAR or fragment thereof comprises the catalytic domain of an ADAR.

31. The method of any one of claims 1-30, wherein the ADAR or fragment comprises the amino acid sequence of SEQ ID NO: 1 or 10 or a sequence having at least 70% sequence identity to SEQ ID NO:1 or 10.

32. The method of any one of claims 1-30, wherein the ADAR or fragment comprises the amino acid sequence of one of SEQ ID NO:1, 3-8, 10, or 11 or an amino acid sequence having at least 70% sequence identity to SEQ ID NO:1, 3-8, 10, or 11.

33. The method of any one of claims 1-32, wherein the cancer comprises melanoma or breast cancer.

34. The method of any one of claim 1-33, wherein the composition is administered by intratumoral or peritumoral injection.

35. The method of any one of claims 1-34, wherein the subject is a human subject.

36. The method of any one of claims 1-35, wherein the ADAR or fragment is from a human ADAR or a derivative thereof.

37. The method of any one of claims 1-36, wherein the method comprises administering a viral particle comprising a nucleic acid encoding a chimeric polypeptide comprising a λN and an ADAR or fragment thereof.

38. The method of claim 37, wherein the viral particle comprises an adeno-associated viral (AAV) particle.

39. The method of claim 38, wherein the AAV serotype is DJ.

40. The method of claim 37, wherein the viral particle comprises an oncolytic viral particle.

41. The method of any one of claim 1-40, wherein the immunotherapy comprises an adjuvant or a nucleic acid encoding an adjuvant.

42. The method of any one of claims 20-41, wherein the immunotherapy comprises viral particles comprising nucleic acids encoding for the immunotherapy.

43. The method of any one of claims 8-42, wherein the ratio of viral particles encoding the ADAR polypeptide or fragment to viral particles encoding the antisense RNA is from about 1:2 to 1:10.

44. The method of claim 43, wherein the ratio is 1:7.

45. The method of any one of claims 6-43, wherein the antisense RNA comprises a antisense RNA and wherein the antisense RNA directs a genomic editing event of a target gene.

46. The method of claim 45, wherein the target gene comprises an oncogene, a tumor suppressor, a metabolic gene, a cytokine, or a growth factor.

47. The method of any one of claims 8-46, wherein the viral particles comprise tumor targeting viral particles.

48. The method of any one of claims 8-47, wherein the viral particles comprise adeno-associated viral particles.

49. The method of any one of claims 1-48, wherein the method further comprises administration of an additional therapy.

50. The method of any one of claims 1-49, wherein the cancer comprises an immunologically cold tumor.

51. The method of any one of claims 1-50, wherein the subject has been determined to have an immunologically cold tumor.

52. A population of viral particles comprising:
(i) viral particles comprising a nucleic acid encoding for a polypeptide comprising a ADAR or fragment thereof; and
(ii) viral particles comprising a nucleic acid encoding for an antisense RNA.

53. A viral particle comprising a nucleic acid encoding for a polypeptide comprising a ADAR or fragment thereof and a nucleic acid encoding for an antisense RNA.

54. The viral particle(s) of claim 52 or 53, wherein the ADAR comprises a hyperactive ADAR or hyperactive ADAR fragment.

55. The viral particles of any one of claims 52-54, wherein the polypeptide further comprises a lambda phage N protein (λN).

56. The viral particles of claim 55, wherein the λN comprises SEQ ID NO:2 or an amino acid sequence with at least 70% sequence identity to SEQ ID NO:2.

57. The viral particles of any one of claims 52-56, wherein the polypeptide further comprises a SNAP tag.

58. The viral particles of any one of claims 54-57, wherein the hyperactive ADAR or fragment has an A-to-G whole genome editing index of greater than 0.5 or an A-to-G Alu element editing index of greater than 6.

59. The viral particles of any one of claims 52-58, wherein the antisense RNA excludes a hairpin.

60. The viral particles of any one of claims 52-59, wherein the antisense RNA excludes a BoxB hairpin.

61. The viral particles of any one of claims 52-60, wherein the antisense RNA is not complimentary to a genomic sequence.

62. The viral particles of any one of claims 52-61, wherein the ADAR or fragment comprises an E448Q substitution.

63. The viral particles of any one of claims 52-62, wherein the ADAR or fragment comprises ADAR2 or a fragment thereof.

64. The viral particles of any one of claims 52-62, wherein the ADAR or fragment thereof comprises the catalytic domain of an ADAR.

65. The viral particles of any one of claims 52-64, wherein the ADAR or fragment comprises the amino acid sequence of SEQ ID NO:1 or 10 or a sequence having at least 70% sequence identity to SEQ ID NO: 1 or 10.

66. The viral particles of any one of claims 52-65, wherein the ADAR or fragment comprises the amino acid sequence of one of SEQ ID NO:1, 3-8, 10, or 11 or an amino acid sequence having at least 70% sequence identity to one of SEQ ID NO:1, 3-8, 10, or 11.

67. The viral particles of any one of claims 52-66, wherein the ADAR or fragment is from a human ADAR or a derivative thereof.

68. The viral particles of any one of claims 52-67, wherein the viral particles comprises adeno-associated viral (AAV) particles or derivatives thereof.

69. The viral particles of claim 68, wherein the AAV serotype is DJ.

70. The viral particles of any one of claims 52-67, wherein the viral particle comprises an oncolytic viral particle.

71. The viral particles of any one of claim 52-70, wherein the viral particles further comprise a nucleic acid encoding an immunotherapy.

72. The viral particles of claim 71, wherein the immunotherapy comprises an immune checkpoint inhibitor (ICI), an immune agonist antibody, or nucleic acids encoding for an immune checkpoint inhibitor or an immune agonist antibody.

73. The viral particles of claim 72, wherein the ICI or immune agonist antibody comprises an anti-CD40 agonistic antibody, anti-PD1 blocking antibody, anti-PDL1 blocking antibody, anti-CTLA4 blocking antibody, or combinations thereof.

74. The viral particles of claim 73, wherein the ICI comprises one or more of Ipilimumab, Tremelimumab, Nivolumab, Pembrolizumab, Atezolizumab, Avelumab, Durvalumab, Spartalizumab, and Cemiplimab.

75. The viral particles of any one of claims 71-74, wherein the immunotherapy comprises pro-inflammatory molecules or nucleic acids encoding for pro-inflammatory molecules.

76. The viral particles of claim 75, wherein the pro-inflammatory molecule comprises IL-2, IL-12, IL-15, or combinations thereof.

77. The viral particles of any one of claims 71-76, wherein the immunotherapy comprises an adjuvant.

78. The viral particles of any one of claims 52-77, wherein the ratio of viral particles encoding the ADAR polypeptide or fragment to viral particles encoding the antisense RNA is from about 1:2 to 1:10.

79. The viral particles of claim 78, wherein the ratio is 1:7.

80. The viral particles of any one of claims 52-79, wherein the antisense RNA comprises a antisense RNA and wherein the antisense RNA directs a genomic editing event of a target gene.

81. The viral particles of claim 80, wherein the target gene comprises an oncogene, a tumor suppressor, a metabolic gene, a cytokine, or a growth factor.

82. The viral particles of any one of claims 52-81, wherein the viral particles comprise tumor targeting viral particles.

83. The viral particles of any one of claims 52-82, wherein the viral particles comprise adeno-associated viral particles.

84. A method for treating cancer in a subject comprising administering the viral particles of any one of claims 52-83 to the subject, wherein the subject is one that has or will receive an immunotherapy.

85. A method for increasing the efficacy of an immunotherapy, the method comprising administering the viral particles of any one of claims 52-83 to the subject, wherein the subject is one that has or will receive an immunotherapy.

86. The method of claim 85, wherein the subject has cancer.

87. The method of any one of claims 84-86, wherein the method further comprises administration of an antisense RNA or a DNA encoding an antisense RNA.

88. The method of claim 87, wherein the antisense RNA excludes a hairpin.

89. The method of claim 87 or 88, wherein the antisense RNA excludes a BoxB hairpin.

90. The method of any one of claims 87-89, wherein the antisense RNA is not complimentary to a genomic sequence.

91. The method of claim 87 or 88, wherein the antisense RNA comprises at least one hairpin.

92. The method of claim 91, wherein the at least one hairpin comprises a BoxB hairpin.

93. The method of claim 91 or 92, wherein the antisense RNA comprises at least two hairpins.

94. The method of any one of claims 12-14, wherein at least one hairpin comprises a nucleotide sequence of GGCCCTGAAAAAGGGCC (SEQ ID NO:9).

95. The method of any one of claims 91-94, wherein the antisense RNA comprises a nucleotide sequence that is complimentary to a cancer neoantigen.

96. The method of claim 95, wherein the neoantigen is selected from Braf p.Val600Glu; KRAS p.Gly12Asp; KRAS p.Gly12Val; TP53 p.Arg175His; KRAS p.Gly12Asp; KRAS p.Gly12Val; HRAS//KRAS/NRAS pGln61Arg; KRAS p.Gly12Val; BRAF p.Val600Glu; and KRAS p.Gly12Asp.

97. The method of any one of claims 87-96, wherein the antisense RNA comprises less than 90 nucleotides.

98. The method of any one of claims 87-97, wherein the antisense RNA comprises less than 60 nucleotides.

99. The method of any one of claims 85-98, wherein the cancer comprises melanoma or breast cancer.

100. The method of any one of claim 85-99, wherein the viral particles are administered by intratumoral or peritumoral injection.

101. The method of any one of claims 85-100, wherein the subject is a human subject.

102. The method of any one of claims 85-100, wherein the method further comprises administration of an additional therapy.

103. The method of any one of claims 85-102, wherein the cancer comprises an immunologically cold tumor.

104. The method of any one of claims 85-103, wherein the subject has been determined to have an immunologically cold tumor.

105. A method for making a viral particle, the method comprising transfecting a packing cell with a nucleic acid encoding for a polypeptide comprising a nucleic acid encoding for an antisense RNA and/or a nucleic acid encoding for an ADAR or fragment thereof; and collecting virus produced from the packaging cell.

106. The method of claim 105, wherein the ADAR or fragment comprises a hyperactive ADAR.


SEQUENCE LISTING (11 sequences)

SEQ ID NO: 1 - protein, 405 aa, synthetic construct
LHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPLV

SEQ ID NO: 2 - protein, 21 aa, synthetic construct
NARTRRRERRAEKQAQWKAAN

SEQ ID NO: 3 - protein, 701 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 4 - protein, 733 aa, synthetic construct
MASSTTGPLHMGTFFSIMGRRYKRRRKKRSERKDRNSLRQSRNPQKYFAMDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCSLPLTTLQDYQAQRVP

SEQ ID NO: 5 - protein, 674 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKVH

SEQ ID NO: 6 - protein, 741 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 7 - protein, 714 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKVH

SEQ ID NO: 8 - protein, 674 aa, synthetic construct; identical to SEQ ID NO: 5

SEQ ID NO: 9 - protein, 17 aa, synthetic construct
GGCCCTGAAAAAGGGCC

SEQ ID NO: 10 - protein, 405 aa, synthetic construct
LHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTPLV

SEQ ID NO: 11 - protein, 701 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP